(19) United States
(12) Patent Application Publication
(10) Pub. No.: US 2025/0125037 A1
(43) Pub. Date: Apr. 17, 2025

(54) METHOD AND DEVICE FOR PROVIDING CUSTOMIZED DIET

(71) Applicant: STAYJUNGLE CO., LTD., Seoul (KR)

(72) Inventor: Eun-Jung KIM, Seoul (KR)

(21) Appl. No.: 18/691,700

(22) PCT Filed: Sep. 6, 2022
(86) PCT No.: PCT/KR2022/013363

§ 371 Date: Mar. 13, 2024

(30) Foreign Application Priority Data
Sep. 14, 2021 (KR) 10-2021-0122686

(51) Int. Cl. G16H 20/60

(52) U.S. Cl. CPC G16H 20/60

(57) ABSTRACT

A method for providing a customized diet, according to one embodiment of the present invention, comprises the steps of: receiving food information from a food seller; deriving nutrition data of food on the basis of the food information; and providing a customized diet to a consumer on the basis of the nutrition data and consumer data.

### TECHNICAL FIELD

The present disclosure relates generally to technology for providing a customized diet to a matching consumer based on information provided from a food seller or supplier.

More particularly, the present disclosure relates to technology that generates a diet pattern based on the lifestyle information and food preference information of each consumer and that places suitable food items among food items input by a seller into the diet pattern based on the food preference information and health information of the consumer.

### BACKGROUND ART

Recently, as consumers' interest in health increases, interest in diets directly related to health is also increasing.

As interest in health care and maintaining immunity rises, the market size for health functional food and nutritional supplements is increasing, and consumers' interest in how to plan their daily diets is also increasing.

For this, there are provided a service that automatically provides nutrient and calorie information based on Artificial Intelligence (AI) when a diet is photographed, a service that recommends a best food type when each consumer inputs information such as health and disease, and the like.

However, in such conventional services, when each consumer inputs information, the information is analyzed and then diet information is merely provided, thus requiring a separate procedure in order to purchase products.

Therefore, there is urgently required technology for providing a customized diet to each consumer based on information received from a supplier or seller, and matching the consumer with the seller.

### DISCLOSURE

### Technical Problem

An object of the present disclosure is to provide a customized diet to a consumer based on information input by a supplier or a seller and match the consumer with the seller.

Another object of the present disclosure is to calculate preference scores of food items by utilizing information input by a supplier or a seller and consumer information, and provide a diet composed of main food and side food based on the preference scores.

A further object of the present disclosure is to provide a customized diet configured depending on the lifestyle, diet purpose, and taste of a consumer.

### Technical Solution

A method for providing a customized diet according to an embodiment of the present disclosure to accomplish the above objects includes receiving food information from a food seller, deriving nutritional data of food based on the food information, and providing a customized diet to a consumer based on the nutritional data and consumer data.

Here, providing the customized diet to the consumer may include calculating preference scores of respective food items based on the consumer data, generating a diet pattern based on a preferred dish type and daily meal count information, and placing the food items in the diet pattern based on the calculated scores.

Here, placing the food items in the diet pattern may include placing main food in the food pattern, and placing multiple side food items having ingredients different from ingredients of the main food in the diet pattern.

Here, the preference scores may be calculated as higher scores as more efficacy-valid ingredients corresponding to health information of the consumer are contained, more preferred ingredients of the consumer are included, and less avoided ingredients of the consumer are contained.

Here, providing the customized diet to the consumer may include providing nutritional information corresponding to the customized diet and efficacy information corresponding to ingredients of the customized diet, together with the customized diet.

Here, providing the customized diet to the consumer may further include providing an image in which elements of respective layers corresponding to the customized diet are connected to each other, together with the customized diet.

Here, the layers may include a layer corresponding to the diet, a layer corresponding to the ingredients, a layer corresponding to the nutritional information, and a layer corresponding to the efficacy information.

Here, the method may further include providing a report on nutritional information at a preset period based on the provided customized diet.

Here, the report may include calorie information consumed during the preset period, insufficient nutritional information, and ingredient information corresponding to the insufficient nutritional information.

Here, the food information may include food or service information of the food seller, store location information, a food type, a food taste, ingredient information, or a cooking method.

Here, the consumer data may include health information, information about a preferred ingredient and an avoided ingredient, a preferred dish type, a daily meal count, or a customized diet service usage period of the consumer.

Further, a device for providing a customized diet according to an embodiment of the present disclosure to accomplish the above objects includes one or more processors, and execution memory configured to store at least one or more programs that are executed by the one or more processors, wherein the at least one or more programs include instructions for performing receiving food information from a food seller, deriving nutritional data of food based on the food information, and providing a customized diet to a consumer based on the nutritional data and consumer data.

Here, providing the customized diet to the consumer may include calculating preference scores of respective food items based on the consumer data, generating a diet pattern based on a preferred dish type and daily meal count information, and placing the food items in the diet pattern based on the calculated scores.

Here, placing the food items in the diet pattern may include placing main food in the food pattern, and placing multiple side food items having ingredients different from ingredients of the main food in the diet pattern.

Here, the preference scores may be calculated as higher scores as more efficacy-valid ingredients corresponding to health information of the consumer are contained, more preferred ingredients of the consumer are included, and less avoided ingredients of the consumer are contained.

Here, providing the customized diet to the consumer may include providing nutritional information corresponding to the customized diet and efficacy information corresponding to ingredients of the customized diet, together with the customized diet.

Here, providing the customized diet to the consumer may further include providing an image in which elements of respective layers corresponding to the customized diet are connected to each other, together with the customized diet.

Here, the layers may include a layer corresponding to the diet, a layer corresponding to the ingredients, a layer corresponding to the nutritional information, and a layer corresponding to the efficacy information.

Here, the programs may further include an instruction for performing providing a report on nutritional information at a preset period based on the provided customized diet.

Here, the report may include calorie information consumed during the preset period, insufficient nutritional information, and ingredient information corresponding to the insufficient nutritional information.

Here, the food information may include food or service information of the food seller, store location information, a food type, a food taste, ingredient information, or a cooking method.

Here, the consumer data may include health information, information about a preferred ingredient and an avoided ingredient, a preferred dish type, a daily meal count, or a customized diet service usage period of the consumer.

### Advantageous Effects

According to the present disclosure, a customized diet may be provided to a consumer based on information input by a supplier or a seller, and the consumer may be matched with the seller.

Further, the present disclosure may calculate preference scores of food items by utilizing information input by a supplier or a seller and consumer information, and may provide a diet composed of main food and side food based on the preference scores.

Furthermore, the present disclosure may provide a customized diet configured depending on the lifestyle, diet purpose, and taste of a consumer.

### DESCRIPTION OF DRAWINGS

FIG. 1 is a flowchart illustrating a method for providing a customized diet according to an embodiment of the present disclosure;

FIG. 2 is a flowchart illustrating in detail step S130 of providing a customized diet to a consumer;

FIG. 3 is a diagram illustrating an example of food data;

FIG. 4 is a diagram illustrating a screen provided to a consumer in a method for providing a customized diet according to an embodiment of the present disclosure;

FIG. 5 is a diagram illustrating an example of food ingredient data in a method for providing a customized diet according to an embodiment of the present disclosure:

FIG. 6 is a diagram illustrating a screen provided to a seller in a method for providing a customized diet according to an embodiment of the present disclosure;

FIG. 7 is a diagram illustrating the results of deriving nutritional information and efficacy information based on information input in FIG. 6; and

FIG. 8 is a diagram illustrating the configuration of a computer system according to an embodiment.

### BEST MODE

Advantages and features of the present disclosure and methods for achieving the same will be clarified with reference to embodiments described later in detail together with the accompanying drawings. However, the present disclosure is capable of being implemented in various forms, and is not limited to the embodiments described later, and these embodiments are provided so that this disclosure will be thorough and complete and will fully convey the scope of the present disclosure to those skilled in the art. The present disclosure should be defined by the scope of the accompanying claims. The same reference numerals are used to designate the same components throughout the specification.

It will be understood that, although the terms “first” and “second” may be used herein to describe various components, these components are not limited by these terms. These terms are only used to distinguish one component from another component. Therefore, it will be apparent that a first component, which will be described below, may alternatively be a second component without departing from the technical spirit of the present disclosure.

The terms used in the present specification are merely used to describe embodiments, and are not intended to limit the present disclosure. In the present specification, a singular expression includes the plural sense unless a description to the contrary is specifically made in context. It should be understood that the term “comprises” or “comprising” used in the specification implies that a described component or step is not intended to exclude the possibility that one or more other components or steps will be present or added.

Unless differently defined, all terms used in the present specification can be construed as having the same meanings as terms generally understood by those skilled in the art to which the present disclosure pertains. Further, terms defined in generally used dictionaries are not to be interpreted as having ideal or excessively formal meanings unless they are definitely defined in the present specification.

Hereinafter, embodiments of the present disclosure will now be described in detail with reference to the accompanying drawings, identical reference numerals are assigned to indicate identical or similar elements in descriptions made with reference to the drawings, and overlapping descriptions thereof will be omitted.

A method for providing a customized diet according to an embodiment of the present disclosure collects personal health information, lifestyle information, and taste information through a simple conversation with a chatbot, matches the food of a local restaurant with the collected information, and provides matching information.

Further, by means of the information about the provided food, the food is considered to have been consumed after purchase, and consumed dietary data is automatically stored, whereby a report in which nutritional status is analyzed may be periodically provided through the chatbot.

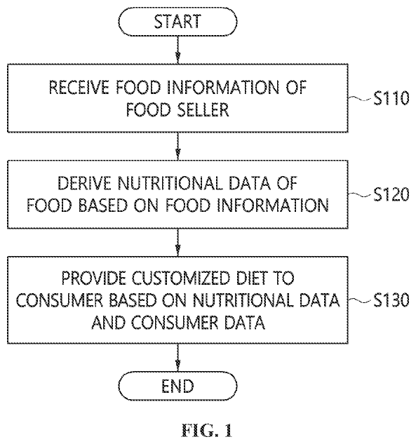

FIG. 1 is a flowchart illustrating a method for providing a customized diet according to an embodiment of the present disclosure.

Referring to FIG. 1, the diet provision method performed by a device for providing a customized diet receives food information from a food seller or supplier at step S110.

Here, step S110 of receiving the food information may correspond to the step of receiving food information input by the food seller using a seller terminal.

Here, step S110 of receiving the food information from the food seller or supplier may be performed according to a preset scheme.

For example, the food seller may input food information to a service screen output via the seller terminal according to a preset scheme.

Here, the food seller may be a concept that includes a food seller through a restaurant, a delivery food seller, a catering company, a packaged food seller, etc.

When the food seller corresponds to the catering company, the food seller may input pieces of information that can be selected by the consumer, such as the presence or non-presence of a chef certification, culinary experience, an hourly rate, and specialties.

Here, the food seller may input local information including information, such as the location information of a restaurant and a delivery service available area, for the purpose of enabling a delivery service, a food pickup service or a consumer dine-in service.

Additionally, the food seller may input overall information related to operation, such as the operating hours, delivery hours, and break time of the restaurant or service.

Moreover, the food seller may additionally input facility information, such as restaurant address information, business license information, and store photos.

Here, the food seller may input information related to food items actually being sold.

Here, the food seller may input a recipe for a specific purpose as well as food items actually being sold.

For example, the recipe for the specific purpose may correspond to a ketogenic recipe, a low carbohydrate-high fat recipe, a diet recipe, and a food recipe for patients with a specific disease.

Here, the customized diet provision device according to the embodiment of the present disclosure may provide an input screen for data acquisition.

Here, the input screen for data acquisition may correspond to the form of an application, a webpage, the chatbot of a mobile messenger, or an email.

Here, the food seller may enter the type (genre) of food desired to be input.

For example, food types may be categorized into Korean food, Chinese food, Japanese food, Western food, etc., or may be categorized into Korean food (e.g., stews, hot pots, grilled dishes, braised dishes, etc.), Western food (e.g., salads, pasta dishes, steak dishes, etc.), one-dish meals (e.g., cold noodles, curry, Seolleongtang (Ox Bone Soup), a pork cutlet, etc.), street food, etc. However, the food types may be variously set depending on the purpose of service desired to be provided, and are not limited to the above-examples.

Furthermore, the food seller may input main ingredients for setting diet names.

Here, the number of main ingredients that can be input may correspond to a preset number. For example, the number of main ingredients that can be input may be set to 3, and the maximum number of main ingredients may be set in this way, with the result that the efficacy of ingredients may be more clearly derived.

Furthermore, the food seller may input major and minor ingredients for calculating the calories of the input food. In this case, the quantities of both the major and minor ingredients that are put may be input together with the major and minor ingredients.

Furthermore, the food seller may input seasonings, condiments, toppings, etc. to determine the degree of the taste (flavor) of the input food.

Here, the degree of taste may be classified into sweet, salty, spicy, sour, and the like, but the scope of the present disclosure is not limited thereto.

When the food information of the seller is received at step S110, nutritional data of the input food may be derived based on the food information at step S120.

That is, the customized diet provision method according to the embodiment of the present disclosure may derive nutritional component information and efficacy information by inputting the food information depending on a preset algorithm.

In this case, the step of deriving the nutritional data of the input food based on the food information may be performed by utilizing prestored food ingredient information or by applying a deep learning network.

In this case, the food ingredient information may be stored in the form of a mapping table, and thus efficacy information and nutritional information corresponding to a specific food ingredient may be derived.

In this case, the deep learning network may correspond to various types of network structures such as a Convolutional Neural Network (CNN) and a Generative Adversarial Network (GAN).

When the nutritional data of food is derived, a customized diet is provided to the consumer based on the nutritional data and consumer data at step S130.

Here, the consumer data may correspond to data prestored in a server or correspond to data input by the consumer in real time. That is, the customized diet may be provided to a consumer matching the food information of the seller based on the prestored consumer data, or may be provided with respect to the information input by the consumer in real time.

Here, step S130 of providing the customized diet to the consumer may provide the customized diet based on location information.

For example, food information of a store located within a preset distance from the location of the consumer may be provided, or, alternatively, information about food available for a delivery or catering service to the location of the consumer may be provided.

That is, the consumer may easily visit the store of the corresponding seller or utilize the delivery or catering service by providing the customized diet service based on the locations of the food seller and the consumer.

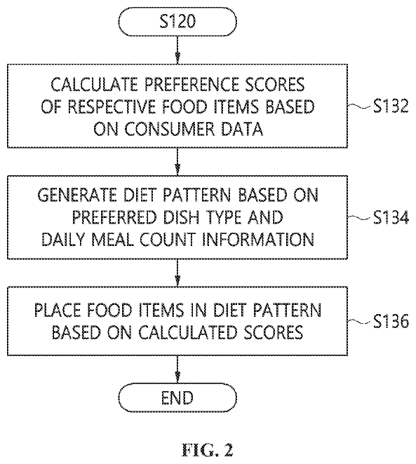

FIG. 2 is a flowchart illustrating in detail step S130 of providing the customized diet to the consumer.

Referring to FIG. 2, step S130 of providing the customized diet to the consumer may include step S132 of calculating preference scores of respective food items based on the consumer data, step S134 of generating a diet pattern based on a preferred dish type and daily meal count information, and step S136 of placing the food items into the generated diet pattern based on the calculated scores.

That is, the customized diet provision method may configure the customized diet for the consumer by using the consumer data and the nutritional data as input.

Here, the consumer data may include health data including information about health conditions desired to be managed, such as a blood sugar level, blood pressure, a musculoskeletal system, and a nervous system, preference data including information about preferred ingredients, avoided ingredients, and preferred dish styles (e.g., Korean food, Western food, etc.), and lifestyle data including information about a diet program subscription period, a daily meal count, etc.

Here, in relation to the nutritional data, the food information received at step S110 may include ingredients used for each dish, nutritional components contained in each ingredient, and effects corresponding to respective nutritional components.

Here, the preference scores may be calculated as higher scores as more efficacy-valid ingredients corresponding to the health information of the consumer are contained, more preferred ingredients of the consumer are included, and less avoided ingredients of the consumer are contained.

However, a method of calculating the preference scores is not limited thereto, and may be variously implemented depending on data that is used and the purpose of the diet.

Here, step S134 of generating the diet pattern may be performed by utilizing the lifestyle data and the preferred dish style information.

For example, for a consumer who prefers the Western food over Korean food, the diet pattern such as (Korean food, Western food, Western food, Korean food, Western food, Western food, . . . ) may be generated.

In addition, a customized diet pattern may be provided to the consumer by utilizing data about a daily meal count, data about whether the consumer has breakfast, etc.

Here, the diet pattern may be generated to correspond to a preset period such as a day, a week, or a month.

When the diet pattern is generated, food items may be placed in the diet pattern based on the preference scores at step S136.

Here, step S136 of placing the food items may include the step of placing main food in the diet pattern and the step of placing multiple side food items having ingredients different from those of the main food in the diet pattern.

That is, after only food items corresponding to the main food are extracted at step S132, the food items may be placed in the diet pattern, and multiple side food items having ingredients and principal taste components that do not overlap those of the main dish may be combined with the placed main food. Here, the number of side food items may be set to various numbers such as 2 and 3.

Here, step S130 of providing the customized diet to the consumer may provide the nutritional information corresponding to the customized diet and efficacy information corresponding to the ingredients of the customized diet together with the customized diet.

Although not illustrated in FIG. 1, the customized diet provision method according to an embodiment of the present disclosure may further include the step of providing a report on the nutritional information at a preset period based on the customized diet.

Here, the report on the nutritional information may include information about calories consumed during the preset period, insufficient nutritional information, and ingredient information corresponding to the insufficient nutritional information.

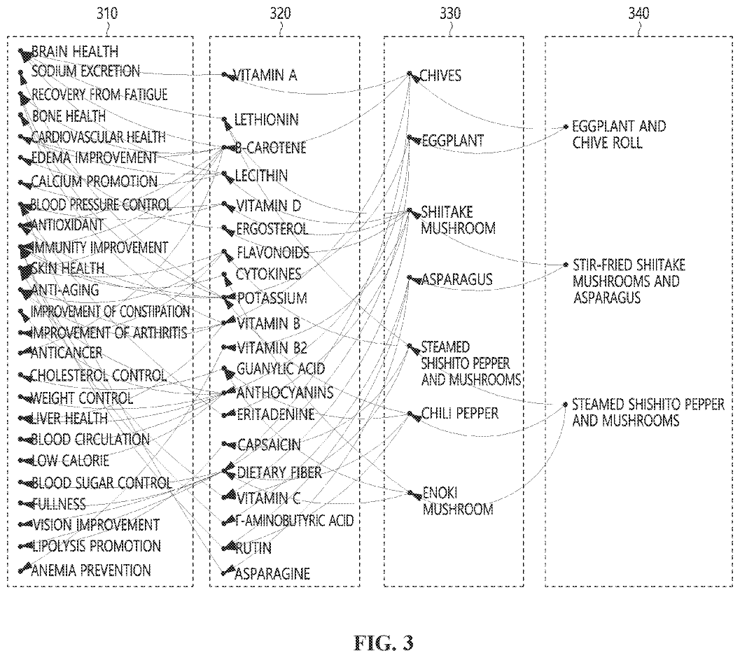

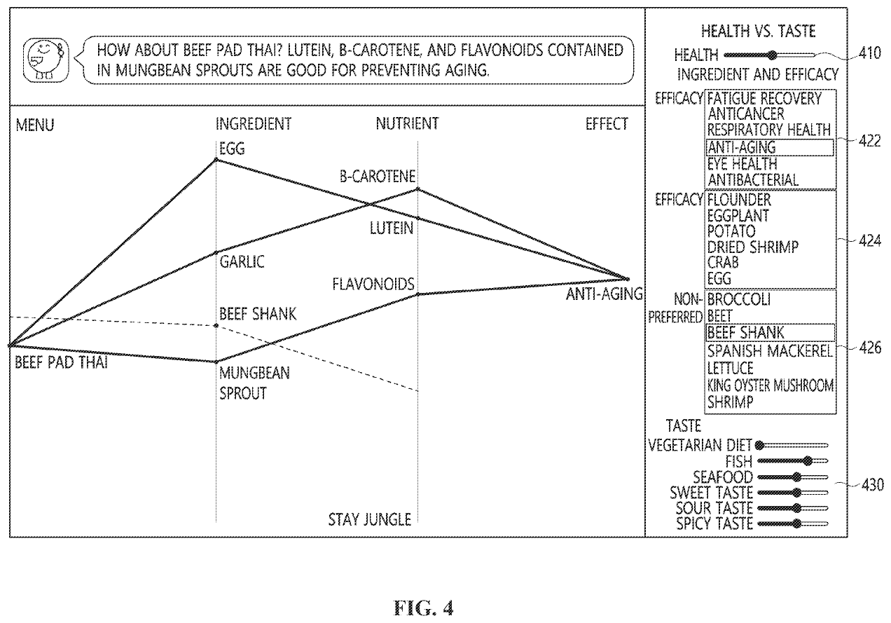

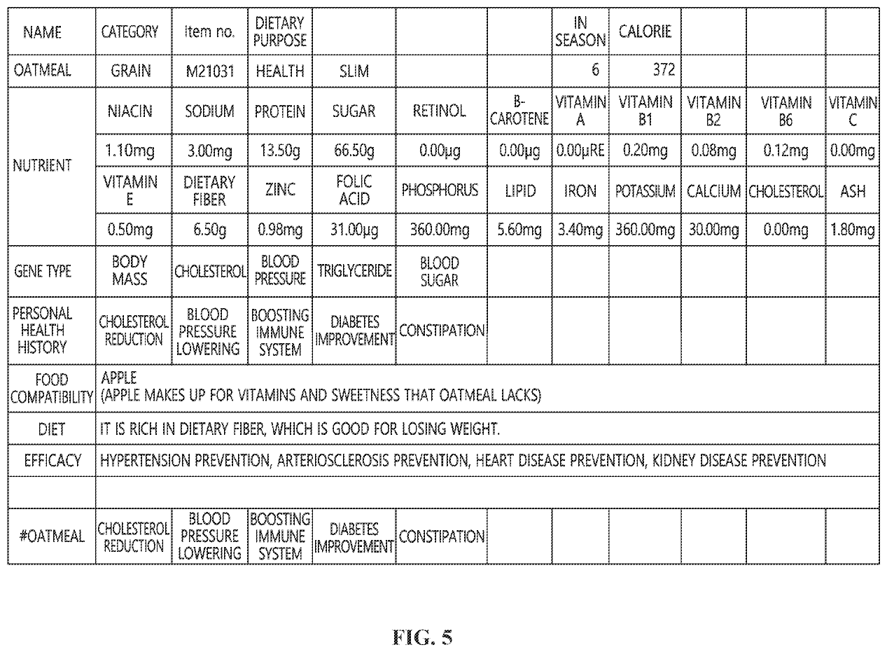

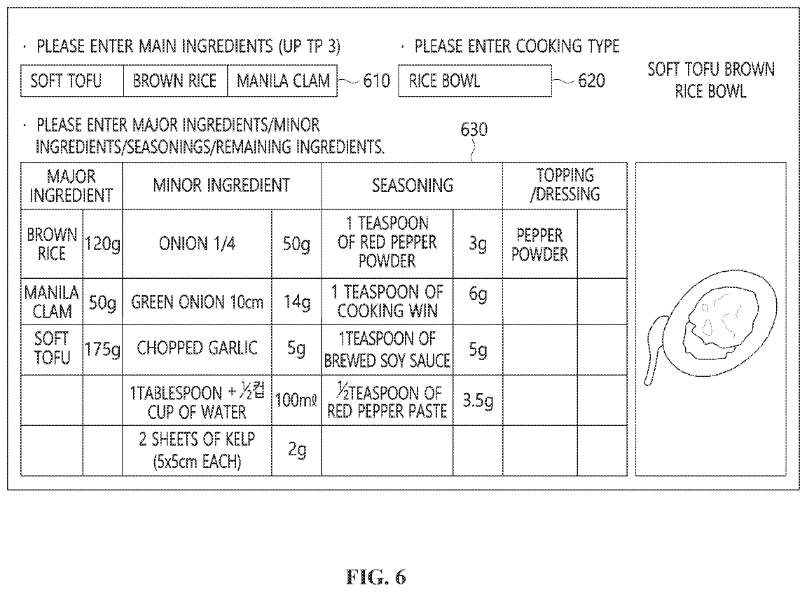

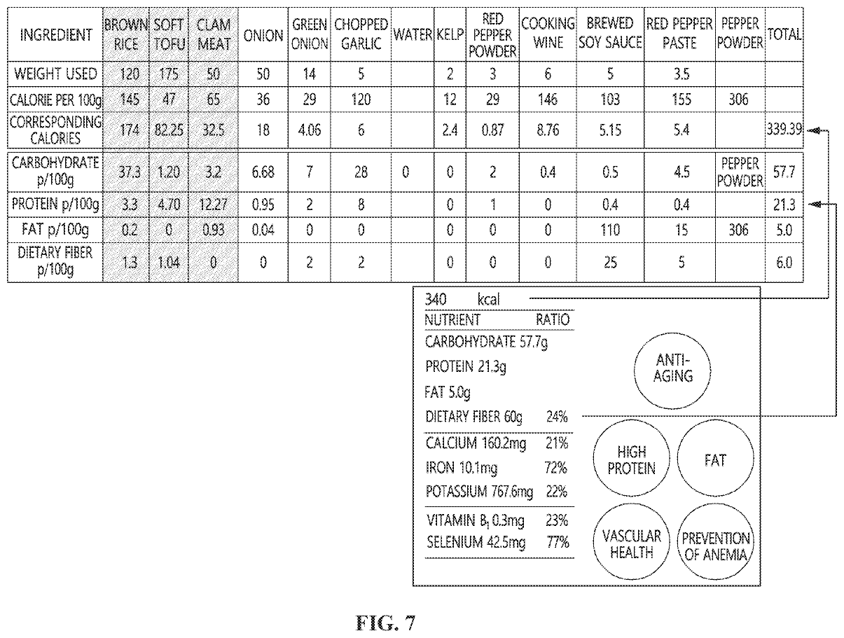

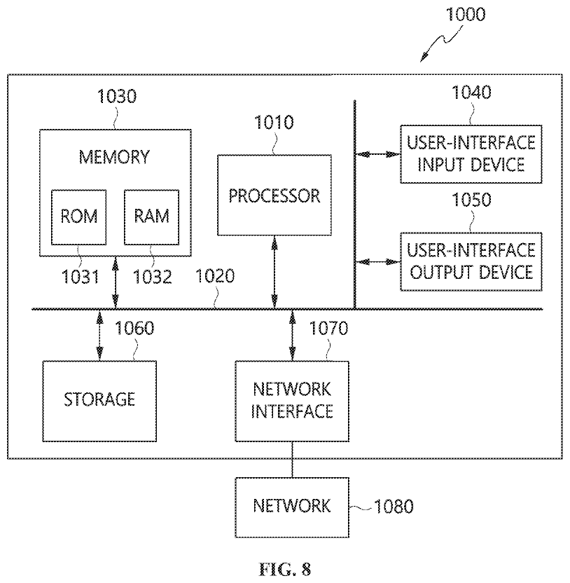

FIG. 3 is a diagram illustrating an example of food data.
Referring to FIG. 3, food data may include efficacy data 310 based on nutritional components, nutritional component data 320 corresponding to each ingredient, food ingredient data 330 used in each dish, and dish data 340.
When a diet is input using the above-described data, expected efficacy may be reported, or when the consumer inputs desired efficacy, food ingredients or dishes corresponding to the desired efficacy may be recommended. Further, the above-described data may be utilized to calculate the preference scores of the consumer for food.
FIG. 4 is a diagram illustrating a screen provided to a consumer in a method for providing a customized diet according to an embodiment of the present disclosure.
Referring to FIG. 4, the consumer may select the relative importance of health and taste in a health selection field 410.
Further, the consumer may select efficacy desired to be obtained through a diet, such as fatigue recovery, anticancer, respiratory health, anti-aging, and eye health in an efficacy selection field 422.
Furthermore, the consumer may select preferred ingredients and non-preferred ingredients in a preferred ingredient selection field 424 and a non-preferred ingredient selection field 426, respectively.
Furthermore, taste levels for a vegetarian diet, fish, seafood, sweet taste, sour taste, spicy taste, etc. may be established in a taste selection field 430.
When the consumer selects ingredients, efficacy, taste, etc., a diet (menu) may be recommended in the form of a chatbot or an image, as illustrated in FIG. 4.
Referring to FIG. 4, the consumer selected anti-aging as efficacy and selected beef shank as a non-preferred ingredient.
As described above, the results of a menu derivation process based on the consumer's selection, food ingredients, nutntional information, and efficacy information may be presented in the form of an image so that the results are recognized at a glance.
Further, in a form similar to the chatbot conversation shown in FIG. 4, menus, ingredients included in the menus, nutrients contained in the ingredients, and the corresponding efficacy based on the nutrients may be provided together.
FIG. 5 is a diagram illustrating an example of food ingredient data in a method for providing a customized diet according to an embodiment of the present disclosure.
Referring to FIG. 5, ingredients may be categorized into grains, vegetables, fruits, fish, meat, seafood, seasonings, etc., wherein oatmeal corresponds to the category of grains.
Further, for the food ingredient data according to an embodiment of the present disclosure, calories, nutrients, and efficacy for respective food ingredients may be classified into a gene type and a personal health history type.
Furthermore, the ingredients may also be classified depending on diet purposes such as diet (Slim), health management (Health), anti-aging (Young), and brain health (Smart).
Suggested ingredients may be recommended or information thereof may be provided in conformity with personal health conditions by utilizing the ingredient data configured, as illustrated in FIG. 5. Furthermore, customized recipes and meals using customized ingredients may be provided.
Furthermore, consumed food information may be automatically stored, and body changes corresponding thereto may be monitored.
FIG. 6 is a diagram illustrating a screen provided to a seller in a method for providing a customized diet according to an embodiment of the present disclosure;
The screen provided to the seller may include a main ingredient input field 610 into which main ingredients can be input, a cooking type input field 620 into which cooking types can be input, and an additional information input field 630.
Here, a food seller may individually upload pictures of input food.
When the food seller inputs information about food, nutrients and calories may be derived and provided based on food ingredient data.
Here, calories and nutrients such as in sodium content are derived, after which food matching a preset condition may be selected as health food.
FIG. 7 is a diagram illustrating the results of deriving nutritional information and efficacy information based on information input in FIG. 6.
Referring to FIG. 7, nutritional information and efficacy information of major ingredients, minor ingredients, seasonings, and topping, input in FIG. 6, may be known.
FIG. 8 is a diagram illustrating the configuration of a computer system according to an embodiment.
A customized diet provision device according to an embodiment may be implemented in a computer system 1000 such as a computer-readable storage medium.
The computer system 1000 may include one or more processors 1010, memory 1030, a user interface input device 1040, a user interface output device 1050, and storage 1060, which communicate with each other through a bus 1020. The computer system 1000 may further include a network interface 1070 connected to a network 1080. Each processor 1010 may be a Central Processing Unit (CPU) or a semiconductor device for executing programs or processing instructions stored in the memory 1030 or the storage 1060. Each of the memory 1030 and the storage 1060 may be a storage medium including at least one of a volatile medium, a nonvolatile medium, a removable medium, a non-removable medium, a communication medium or an information delivery medium, or a combination thereof. For example, the memory 1030 may include Read-Only Memory (ROM) 1031 or Random Access Memory (RAM) 1032.
A device for providing a customized diet according to an embodiment of the present disclosure may include one or more processors, and execution memory configured to store at least one or more programs that are executed by the one or more processors.
Here, the at least one or more programs may include instructions for performing receiving food information from a food seller, deriving nutritional data of food based on the food information, and providing a customized diet to a consumer based on the nutritional data and consumer data.
Here, the food information may include food or service information of the food seller, store location information, a food type, a food taste, ingredient information, or a cooking method.
Here, the consumer data may include health information, information about a preferred ingredient and an avoided ingredient, a preferred dish type, a daily meal count, or a customized diet service usage period of the consumer
Here, providing the customized diet to the consumer may include calculating preference scores of respective food items based on the consumer data, generating a diet pattern based on a preferred dish type and daily meal count information included in the consumer data, and placing the food items in the diet pattern based on the calculated scores.
Here, placing the food items in the diet pattern may include placing main food in the food pattern, and placing multiple side food items having ingredients different from ingredients of the main food in the diet pattern.
Here, the preference scores may be calculated as higher scores as more efficacy-valid ingredients corresponding to health information of the consumer are contained, more preferred ingredients of the consumer are included, and less avoided ingredients of the consumer are contained.
Here, providing the customized diet to the consumer may include providing nutritional information corresponding to the customized diet and efficacy information corresponding to ingredients of the customized diet, together with the customized diet.
Here, providing the customized diet to the consumer may include providing an image in which elements of respective layers corresponding to the customized diet are connected to each other, together with the customized diet.
Here, the layers include a layer corresponding to the diet, a layer corresponding to the ingredients, a layer corresponding to the nutritional information, and a layer corresponding to the efficacy information.
Here, the programs may further include an instruction for performing providing a report on nutritional information at a preset period based on the provided customized diet.
Here, the report may include calorie information consumed during the preset period, insufficient nutritional information, and ingredient information corresponding to the insufficient nutritional information.
The particular implementations shown and described herein are illustrative examples of the present disclosure and are not intended to limit the scope of the present disclosure in any way. For the sake of brevity, conventional electronics, control systems, software development, and other functional aspects of the systems may not be described in detail. Furthermore, the connecting lines or connectors shown in the various presented figures are intended to represent exemplary functional relationships and/or physical or logical couplings between the various elements. It should be noted that many alternative or additional functional relationships, physical connections, or logical connections may be present in an actual device. Moreover, no item or component may be essential to the practice of the present disclosure unless the element is specifically described as “essential” or “critical”.
Therefore, the spirit of the present disclosure should not be limitedly defined by the above-described embodiments, and it is appreciated that all scopes of the accompanying claims and equivalents thereof belong to the scope of the spirit of the present disclosure.

1. A method for providing a customized diet, comprising:
receiving food information from a food seller;
deriving nutritional data of food based on the food information; and
providing a customized diet to a consumer based on the nutritional data and consumer data.

2. The method of claim 1, wherein providing the customized diet to the consumer comprises:
calculating preference scores of respective food items based on the consumer data;
generating a diet pattern based on a preferred dish type and daily meal count information in the consumer data; and
placing the food items in the diet pattern based on the calculated scores.

3. The method of claim 2, wherein placing the food items in the diet pattern comprises:
placing main food in the food pattern; and
placing multiple side food items having ingredients different from ingredients of the main food in the diet pattern.

4. The method of claim 2, wherein the preference scores are calculated as higher scores as more efficacy-valid ingredients corresponding to health information of the consumer are contained, more preferred ingredients of the consumer are included, and less avoided ingredients of the consumer are contained.

5. The method of claim 1, wherein providing the customized diet to the consumer comprises:
providing nutritional information corresponding to the customized diet and efficacy information corresponding to ingredients of the customized diet, together with the customized diet.

6. The method of claim 5, wherein:
providing the customized diet to the consumer further comprises:
providing an image in which elements of respective layers corresponding to the customized diet are connected to each other, together with the customized diet, and
the layers include a layer corresponding to the diet, a layer corresponding to the ingredients, a layer corresponding to the nutritional information, and a layer corresponding to the efficacy information.

7. The method of claim 1, further comprising:
providing a report on nutritional information at a preset period based on the provided customized diet,
wherein the report includes calorie information consumed during the preset period, insufficient nutritional information, and ingredient information corresponding to the insufficient nutritional information.

8. The method of claim 1, wherein the food information includes food or service information of the food seller, store location information, a food type, a food taste, ingredient information, or a cooking method.

9. The method of claim 1, wherein the consumer data includes health information, information about a preferred ingredient and an avoided ingredient, a preferred dish type, a daily meal count, or a customized diet service usage period of the consumer.

10. A device for providing a customized diet, comprising:
one or more processors; and
an execution memory configured to store at least one or more programs that are executed by the one or more processors,
wherein the at least one or more programs comprise instructions for performing:
receiving food information from a food seller;
deriving nutritional data of food based on the food information; and
providing a customized diet to a consumer based on the nutritional data and consumer data.

11. The device of claim 10, wherein providing the customized diet to the consumer comprises:
calculating preference scores of respective food items based on the consumer data;
generating a diet pattern based on a preferred dish type and daily meal count information; and
placing the food items in the diet pattern based on the calculated scores.

12. The device of claim 11, wherein placing the food items in the diet pattern comprises:
placing main food in the food pattern; and
placing multiple side food items having ingredients different from ingredients of the main food in the diet pattern.

13. The device of claim 11, wherein the preference scores are calculated as higher scores as more efficacy-valid ingredients corresponding to health information of the consumer are contained, more preferred ingredients of the consumer are included, and less avoided ingredients of the consumer are contained.

14. The device of claim 1, wherein providing the customized diet to the consumer comprises:
providing nutritional information corresponding to the customized diet and efficacy information corresponding to ingredients of the customized diet, together with the customized diet.

15. The device of claim 14, wherein:
providing the customized diet to the consumer further comprises:
providing an image in which elements of respective layers corresponding to the customized diet are connected to each other, together with the customized diet, and
the layers include a layer corresponding to the diet, a layer corresponding to the ingredients, a layer corresponding to the nutritional information, and a laver corresponding to the efficacy information.
